(19) United States
(12) Patent Application Publication
(10) Pub. No.: US 2022/0011316 A1
(43) Pub. Date: Jan. 13, 2022

(54) LIQUID TESTING SYSTEM, DEVICES, AND METHODS

(71) Applicants: Johan M. SPOOR, Montclair, NJ (US); Stephane CAMARROQUE, Montclair, NJ (US); Michael C. JONES, Montclair, NJ (US); James PODER, Montclair, NJ (US); James STAVE, Montclair, NJ (US); Howard Y. BELL, Princeton, NJ (US); Joshua E. COLLINS, Princeton, NJ (US); Paul GUIGUIZIAN, Princeton, NJ (US); Herschel WATKINS, Golden, CO (US); Jacob SOMERSON, Golden, CO (US); Philip SCHREIBER, Golden, CO (US); Timothy MUFF, Golden, CO (US); Laura BUCKNAM, Golden, CO (US)

(72) Inventors: Johan M. SPOOR, Montclair, NJ (US); Stephane CAMARROQUE, Montclair, NJ (US); Michael C. JONES, Montclair, NJ (US); James PODER, Montclair, NJ (US); James STAVE, Montclair, NJ (US); Howard Y. BELL, Princeton, NJ (US); Joshua E. COLLINS, Princeton, NJ (US); Paul GUIGUIZIAN, Princeton, NJ (US); Herschel WATKINS, Golden, CO (US); Jacob SOMERSON, Golden, CO (US); Philip SCHREIBER, Golden, CO (US); Timothy MUFF, Golden, CO (US); Laura BUCKNAM, Golden, CO (US)

(21) Appl. No.: 17/360,219

(22) Filed: Jun. 28, 2021

Related U.S. Application Data
(60) Provisional application No. 63/046,238, filed on Jun. 30, 2020.

(51) Int. Cl. G01N 33/58; G01N 1/40; G01N 33/569; G01N 15/06

(52) U.S. Cl. CPC G01N 33/587; G01N 1/4005; G01N 33/56911; B82Y 35/00; G01N 15/0618; G01N 33/582; G01N 33/56983

(57) ABSTRACT

A testing system and test cartridge for analyzing a sample of water from a water source for specific analyte levels. The test cartridge including a membrane filter that captures a target analyte while allowing a labelled conjugate to permeate through the membrane. The conjugate includes an analyte-specific labelled binding reagent to bind with the target analyte for optical detection. The direct membrane interrogation (i.e., on-filter detection), determines analyte levels without elution of the analyte from a filter thereby improving analyte recovering and assay sensitivity.

### CROSS-REFERENCE TO RELATED APPLICATION

This application claims the benefit of priority of U.S. Provisional Application No. 63/046,238 filed on Jun. 30, 2020.

### TECHNICAL FIELD

The invention relates to water or fluid treatment, sampling, and testing devices. The invention samples and tests water or other fluids for analytes of interest.

### BACKGROUND

Quality testing of liquids, such as water, juice, milk, beer, wine, and others are commonly done by sending out samples to a lab or purchasing test kits and performing the testing in house. However, each of these methods has their shortcomings. Sending samples to a lab takes a lot of time, and test kits are not always as accurate and cannot always differentiate among substances of interest in the water. For example, some test kits may determine bacteria count in general but cannot separate normal bacteria from harmful bacteria such as Escherichia coli (E. coli)) or Legionella pneumophila. Further, some of these harmful substances may affect many water-based uses, including HVAC humidity regulation, agriculture, industrial, and other home uses, in addition to drinking water.

Other water testing devices and methods prepare samples for use by greatly concentrating analytes in the water. For example, the devices use a large water sample to collect and concentrate analytes and elute the concentrated sample from the filter for analysis. Some example devices use filters to trap retentate (e.g., larger particles such as dirt and analytes of interest such as bacteria) and allow the permeate (e.g., water) to pass through the filter. The trapped analytes in the filter are eluted to be collected for analysis of analyte levels in the original sample. These concentration and collection methods generate many inefficiencies. Some of the inefficiencies include longer testing times, lower sensitivity, and a lower percentage of recovery of the trapped analyte from the filter. The concentration and collection methods require longer testing times requiring larger volumes of permeate carrying analytes to concentrate analytes through filtration. The concentration and collection methods have lower sensitivity which requires higher concentrations of analytes in order be detected. These inefficiencies of current designs result in increased testing membrane costs, longer testing times, and increased intervention by those who need the testing results.

### SUMMARY

The methods and devices of the invention include inline water testing devices that are connected to a water source and benchtop testing devices for separate analyzing of water sources. The testing devices comprise an assay and a testing cartridge for analysis of water samples from water sources. The invention includes disposable testing cartridges that filter the water sample to be analyzed for analytes of interest (i.e., target analyte) in the water and are used for direct membrane interrogation. The invention provides improved testing systems for faster, more efficient (e.g., near real-time and higher sensitivity) water testing and need smaller sample sizes at potentially lower analyte levels. The invention further simplifies testing methods of analyte levels by removing the need to elute analytes from filters. The invention completely removes losses from the elution process.

The invention provides assays, which use optically detectable labels in the testing cartridges to determine target analyte levels in the samples of water. The invention provides automated testing of water in systems. The invention provides fluidic paths to filter the water samples, react analyte-specific labelled binding reagents to target analytes captured on the filter, as well as wash the filters of non-specifically absorbed analyte-specific binding reagent for direct membrane interrogation. The invention also provides a conjugate of analyte-specific binding reagent with the optically detectable labels for analysis of analytes captured on the filter. These fluidic paths may each include pumps, valves, and waste containers. The pumps and valves are in communication with a processor and/or controller to force fluids (e.g., water or solutions) through the testing cartridge. In one embodiment of the invention, the flow rate of fluids through the testing cartridge is capped at 30 mL/min. However, the fluid flow rate can be adjusted to allow effective collection of the targeted analyte by the filter, binding of the analyte-specific labelled binding reagent, as well as washing of analyte-specific labelled binding reagent without removal of the collected target analyte capped at 30 mL/min. In one embodiment, the system prevents pressure in the fluidic paths from exceeding a predetermined pressure threshold to prevent damage to the testing cartridge and to prevent false readings.

The invention provides testing cartridges which include a filter membrane. The labels may include colorimetric or phosphor labels including fluorescent molecules and/or particles, such as up-converting nanoparticles. The labels are conjugated to analyte-specific binding reagents, such as antibodies or nucleic acids, that bind specifically to target analytes in the sample. The labels and analyte-specific binding reagents form a conjugate which is introduced to the sample post-filtering after capturing of the target analyte and allowing the permeate to pass through the filter membrane (i.e., hereinafter the “conjugate”). The labels are selected to emit light after absorption of energy. In one example embodiment of the invention, the filter membrane of the testing cartridge has 0.22 μm-sized pores that are larger than the conjugate, but smaller than the target analyte to be captured by the filter membrane. The analyte-specific binding reagents, including antibodies, bind specifically to the target analyte, such as bacteria (e.g., Legionella pneumophila and E. coli). The analyte-specific binding reagents can also bind specifically to target analytes such as viruses, polyfluoroalkyl substances (PFAS), polymers, aggregates, proteins, nucleic acids, toxins, chemical contaminants, and other target analytes. Thus, the water samples are filtered through the membranes and target analytes. For example, bacteria are captured by the filter membrane. The labelled antibody conjugate specifically binds with the captured bacteria on the membrane. Excess, non-specifically bound conjugate is washed from the filter membrane, and the testing site may be dried prior to optical analysis. The assay uses a laser to excite the labelled conjugate remaining on the filter membrane such as conjugated antibodies bound to the bacteria. The labels, once excited by the laser, emit a fluorescence, which is optically detectable. Thus, the presence of fluorescence indicates the presence of the target analyte (such as bacteria, for example) and the amount (intensity) of fluorescence determines the bacteria levels in the water. In some embodiments, the filter membranes include a seal. In some embodiments, conjugate packets in the testing cartridge include a seal.

In some example embodiments of the invention, the testing cartridges are configured to seal in-line with the fluidic paths. The testing cartridge is also configured to provide sufficient filter membrane space. The membrane space is in terms of a surface area, to accurately capture the analytes in the water sample for determination of the analyte levels. Further, the testing cartridges include a sealed barrier to prevent adulteration of the cartridge prior to testing. Additionally, the filter membranes of the testing cartridges may be made of a polyvinylidene fluoride (PVDF), hydrophilic polyester (PETE), nitrocellulose, cellulose acetate, and other materials that meet the system requirements. The filter membrane has larger pores than the conjugate, but smaller pores than the target analyte of interest. In other words, the filter membrane collects and concentrates the target analyte while allowing unbound conjugate to pass through.

For example, when the target analyte is bacteria (e.g., E. coli, Legionella pneumophila, etc.) the pore size of the filter membrane may be 0.22 μm as described above. However, for smaller target analytes, such as viruses, the pore size of the filter membrane must be much smaller, e.g., 15 nm to prevent passing of the viruses through the filter membrane pores. Similarly, the conjugate must also be smaller than the pore size of 15 nm so that unbound conjugate passes through the filter membrane without being captured.

In one embodiment of the invention, a testing device stores many testing cartridges for single use testing of a sample of water. The device dispenses and positions each testing cartridge as testing is needed. The device also positions, as needed, the testing cartridges for drying, excitation using a laser, and optical analysis. In one example embodiment of the invention, the device dispenses a testing cartridge onto a rotating base for filtering the water sample, drying the filter, and measuring analyte levels. In one example embodiment, analyte levels are measured using laser excitation and optical analysis. In one embodiment, the testing cartridge is in a reel including many testing sites separated by hydrophobic regions. The device positions the reel by feeding the testing cartridge into a position for fluidic pathing of the water sample filtering, and further positioning for the excitation process and optical reader.

In some example embodiments of the invention, the testing cartridges accept 3 mL of antibody solution. In some embodiments, the testing cartridges may tolerate 15 psi or less pressure from the fluidic paths. In some embodiments, the testing cartridges are configured to include sufficient cross-sectional flow area to accept a fluidic path flow rate of 10 L/hr.

As used herein, the term “water” is generally used in reference to any subject fluid of interest that may contain target analytes. In other embodiments, the fluids may include human-ingestible fluids such as milk, beer, wine, or other solutions that may need analyte levels determined, such as laboratory buffers or test samples.

As used herein, the term “labels” is generally used in reference to any substance that is used to make an analyte-specific binding reagent optically detectable as a conjugate. More specifically, as used herein, the term “labels” refers to an optically detectable molecule or particle acting as a label that may include colorimetric elements, phosphor particles, fluorescent molecules, and/or other particles, including up-converting nanoparticles that exhibit photon up conversion for better optical detection. The label may be conjugated with analyte-specific binding reagents directed at binding with a target analyte in the water. The analyte-specific binding reagents may also be any other molecule which elicits a response to a targeted analyte in a subject fluid. The label may be an upconverting nanoparticle labelling an antibody in a conjugate.

As used herein, the term “analyte-specific binding reagents” is generally used in reference to any substance that is capable of specifically reacting or binding with a targeted analyte. More specifically, as used herein, the term “analyte-specific binding reagents” refers to an antibodies or nucleic acids for reacting to a particular bacterium. However, the “analyte-specific binding reagents” could refer to any number of reactants that bind specifically to targeted analytes that include bacteria, viruses, particles, chemicals, and other target analytes.

The methods of use of the assays of the invention include loading of a test cartridge to pass a sample of fluid from a fluid source through a filter membrane of the test cartridge. Once the sample is passed, a conjugate of labels and analyte-specific binding reagents is passed through the filter membrane to bind with a target analyte that was captured on the filter membrane. The assay interrogates the sample-passed filter membrane for the labels bound to the target analyte to determine a level of the target analyte in the sample. In some embodiments, the sample-passed filter membranes are prepped for interrogation by washing the sample-passed filter membrane with a solution to remove excess conjugate. In some embodiments, the sample-passed filter membranes are dried prior to interrogation. In some embodiments, the labels of the conjugate bound to the target analytes are excited and optically detected, by emitting light, to determine the level of the target analyte. In some embodiments, the excitable and optically detectable labels are up-converting nanoparticles. Further, in some embodiments, the target analytes are a bacteria or virus. In some embodiments, the labels are selected from a group consisting of colorimetric elements, phosphor molecules, and up-converting nanoparticles.

The assay includes a fluid flow path to provide a sample of fluid from a fluid source to detect analyte levels. The fluid flow path also provides a conjugate of analyte-specific binding reagents with labels to the test cartridge for collection on a filter membrane of the test cartridge. The conjugate collects on the filter membrane by specifically binding to a target analyte previously captured on the filter membrane from passing the sample of fluid. The fluid flow path may include a pump and valve to provide the sample and conjugate to the test cartridge. The assay includes a translational base to position the test cartridge for analysis by the assay. The assay includes an excitation mechanism to excite the labels in the conjugate for optical analysis by an optical detector of the assay. The optical detector detects optical frequencies of the excited labels to determine a level of the target analyte. Further, the assay may include a drying mechanism to dry sample-passed test cartridges.

### BRIEF DESCRIPTION OF THE DRAWINGS

FIG. 1 shows a fluid flow diagram of a testing system in accordance with the invention.

FIG. 2 shows a system diagram of a testing system with a testing cartridge in accordance with the invention.

FIG. 3 shows a side view of a testing system using a testing cartridge for water analyte testing in accordance with the invention.

FIG. 4A shows conjugated labels passed through a filter membrane due to the lack of a target analyte captured on a filter membrane in accordance with the invention.

FIG. 4B shows a filter membrane with conjugate bound to targeted analytes in accordance with the invention.

FIG. 4C shows a filter membrane with conjugate bound to targeted analytes during optical analysis in accordance with the invention.

FIG. 5 shows a filter membrane with conjugate bound to targeted analytes in accordance with the invention.

FIG. 6A shows a perspective view of a loader/storage of testing cartridges in a testing device in accordance with the invention.

FIG. 6B shows a top view of testing cartridges in a loader in accordance with the invention.

FIG. 7 shows a perspective view of a testing device with testing cartridge positioning in accordance with the invention.

FIG. 8 describes a method of using a testing device in accordance with the invention.

### DETAILED DESCRIPTION

The assays of the invention provide a way to test liquids for target analytes.

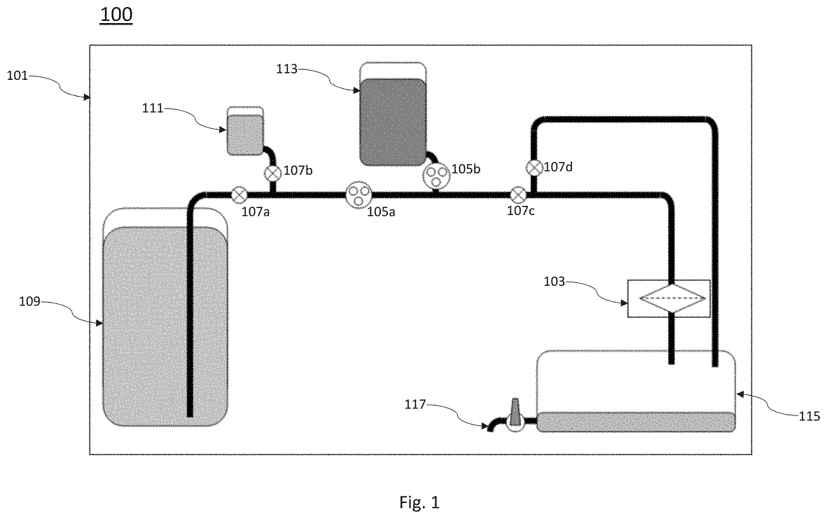

As shown in FIG. 1, a testing system 100 includes fluid flow paths of a testing device 101 configured to accept a testing cartridge 103 to determine analyte levels in a water source. One device 101 includes pumps 105a and 105b and valves 107a-107d configured to be controlled by a processor and regulate flow through the testing system 100. The pumps 105a and 105b may be any type of pump that can accurately provide the fluid needed in the system. In some embodiments, the pumps are peristaltic pumps.

In one embodiment, the testing device 101 includes containers which provide the water sample, such as from water source 109. Container(s) 111, wash container 113, and waste container 115 with a drain 117 are also included in the device 101. Each container includes different solutions for introduction to the filter membrane. These solutions may include analyte-specific binding reagents (i.e., antibody, nucleic acids, aptamers, nanobodies, streptavidin, avidin, proteins, lipoproteins, lectins, carbohydrates, polypeptide ligands of cellular receptors, polynucleotide probes, drugs, antigens, toxins, and the like) and/or other wash solutions, e.g., a solution or buffer with salt or detergent to prevent the conjugate from sticking to the filter membrane. Each container is connected to a valve which may open or close as each fluid from each container is needed. For example, when a sample is needed from water source 109, valve 107a opens, and the sample of water is collected for testing using pump 105a. Similarly, valve 107b is opened for conjugate in the reagent container 111, and pump 105b may be used to pump a wash from wash container 113 throughout the system 100. Further, valve 107c may be opened to provide fluids to the testing cartridge 103, and valve 107d may be opened to provide direct access to the waste container 115. Waste container 115 may include a drain 117 to easily dump waste from the system.

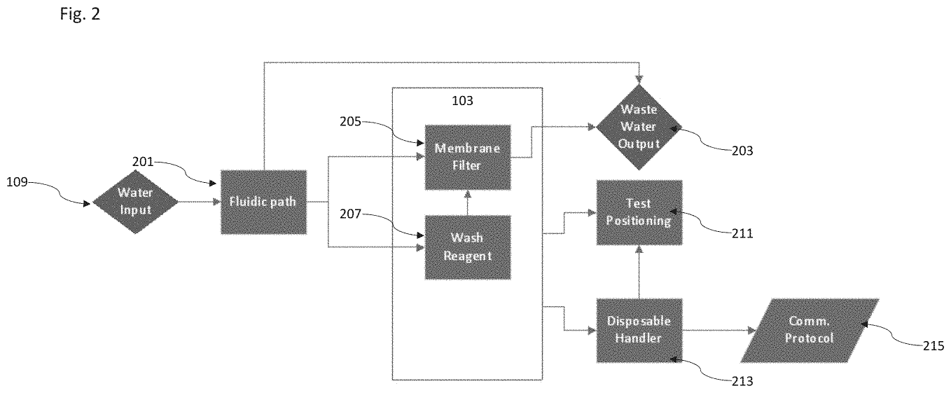

As shown in FIGS. 1 and 2, the testing systems 100 include a water source 109 from which a sample is taken. The sample is introduced into the testing systems 100 through a fluidic pathing 201. The fluidic path, including valves and pumps, provide various fluids to testing cartridges 103 and/or waste output 203. The systems 100 provide the water sample to the testing cartridges 103. The water sample and conjugate may be introduced to the membrane filter 207 of the testing cartridges 103. The testing cartridges 103 may be positioned in the assay for analysis 211 to test for the target analyte. Additionally, the testing cartridges 103 may be disposed through a disposable handler 213 and results and notification of disposal provided to users through a communication protocol 215.

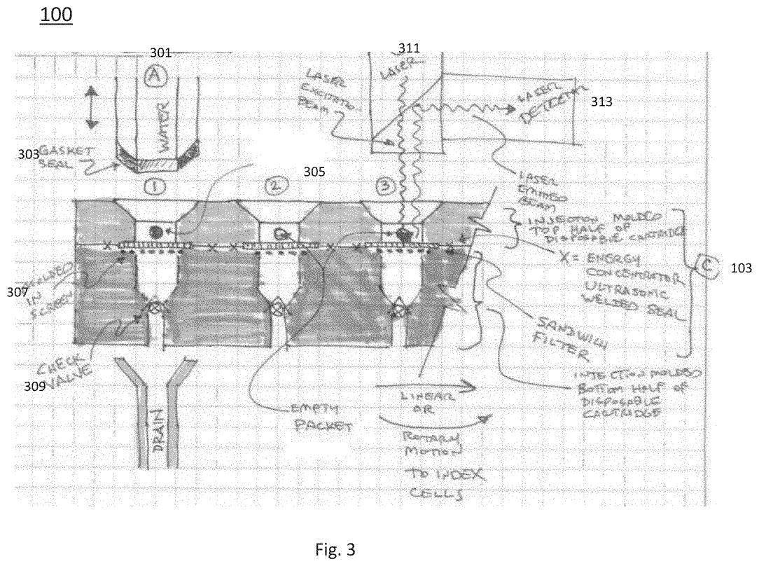

As shown in FIG. 3, the testing systems 100 include providing a wash (e.g., water, wash solution, or other reagent to prevent remaining conjugates from sticking to the membrane filter) through a reagent flow path 301 sealed, with a gasket 303, to the testing cartridges 103 to prevent waste. The testing cartridges 103 include a sealed conjugate packet 305, filter membrane 307, and in some embodiments may include a check valve 309 to create a volume for adsorption, binding, and/or reactions to take place. The conjugate packet 305 (e.g., labelled antibody packet) stores the analyte-specific conjugate in a form and manner that preserves functionality until use (e.g., as a liquid in stabilization buffer, dried or lyophilized), to provide for proper test function and results throughout the shelf-life of the product. The conjugate packet 305 may include a seal for release into the filter membrane 307. The conjugate packet 305 seal is broken with a piston or other piercing mechanism to allow the conjugate to mix with the target analyte for binding. The filter membrane 307 includes pores smaller than the target analyte, but larger than the conjugate. In other embodiments, the conjugate is introduced from a separate reagent container which flows to the testing cartridge 103 through the flow path 301. The bound labels (on the target analyte) may be excited to optically detect analyte levels on the filter membrane 307. The target analyte is collected on the filter membrane prior to introduction of the labelled conjugate. The labelled conjugate reacts/binds to the target analyte remaining on the filter membrane and the amount of remaining conjugate is used to determine the amount of target analyte in the water sample.

In one embodiment, the labelled conjugate may be bound with the sample-passed filter membrane 307 by mixing of the conjugate with the sample-passed filter membrane. The mixing may be through directing flow of the conjugate forward and reverse through the sample-passed filter membrane multiple times to ensure more binding of the conjugate to the target analyte. For example, E. coli or Legionella pneumophila bacteria in a water sample filtered through the filter membrane 307 may bind to labelled antibodies by repeated mixing with a sufficient amount of the labelled antibodies. The testing cartridges 103 may then be washed with water, wash solution, or other reagent to remove excess conjugate in the filter membrane 307 and to remove other chemicals or biologics which may adulterate the test. The testing cartridges 103 may then be purged of fluids with air.

In other embodiments, the water samples are provided to the filter membranes 307 through flow paths 301, to filter and collect targeted analytes. The conjugates may be provided by either the packets 305 or the flow paths 301. The target analytes on the filter membranes 307 react to the conjugate, e.g., labelled antibodies bind to the E. coli or Legionella pneumophila bacteria and that are captured by the filter membranes. The filter membranes 307 are then washed to remove any excess conjugate and set to dry for optical analysis.

In some embodiments, the testing cartridges 103 may then be placed in a dry position to allow drying of the concentrated analytes, e.g., when using up-converting nanoparticles. Drying may aid in optical analysis of the up-converting nanoparticles. In one embodiment, the drying position may include drying mechanisms, such as heaters and fans which do not affect and/or denature the labelled analytes. Once dry, the testing cartridges 103 may be positioned for analysis and disposal. A laser 311 is used to excite the captured labels bound to the analytes and optical analysis through optical detection 313 of the excited nanoparticles provides a concentration level of the target analyte.

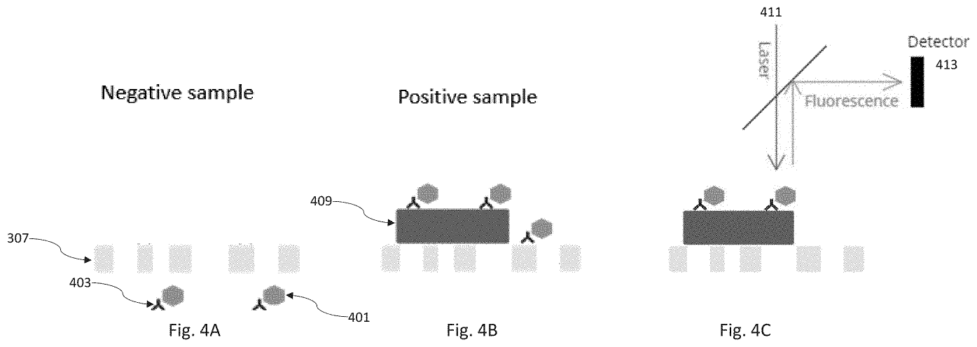

As shown in FIGS. 4A-4C, the optically detectable labels 401 and analyte-specific binding reagents 403, e.g., antibodies are captured on a filter membrane 307 after binding to the target analyte. Those antibodies that do not bind to the target analyte pass through the filter membrane 307. Prior to introduction of the conjugate, when a water sample is introduced if the sample does not contain the targeted analyte, such as in FIG. 4A, the conjugate, e.g., labels 401 with analyte-specific binding reagents 403, have not reacted to a target analyte captured by the filter membrane 307 and simply pass through the filter membrane 307. Washing with a water, wash solution, or reagent further confirms that little to no reaction has occurred between target analyte and the conjugate, since the conjugate is unable to be collected by the filter membrane 307 which includes larger pores than the conjugate.

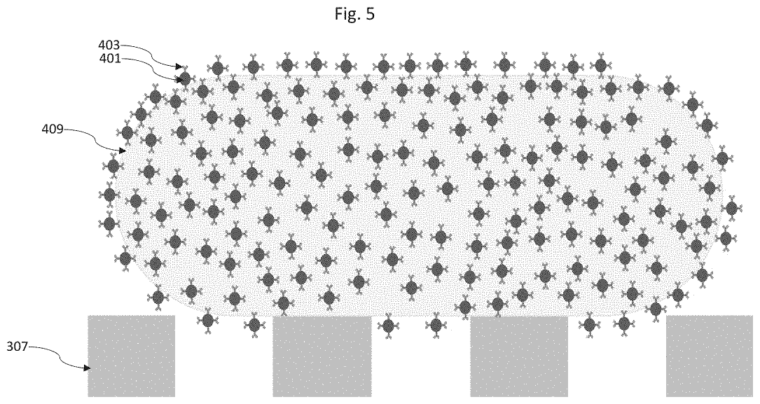

In FIG. 4B, the labels 401 and analyte-specific binding reagents 403 bind to a target analyte 409 in the water sample and remain trapped on the filter membrane 307. Additionally, as shown in FIG. 5, in one embodiment, the labels 401 are 0.02 μm, the antibodies are 0.012 μm, the target analyte 409 (i.e., bacterium cell) is 1 μm, and the filter membrane 307 has a pore size of 0.22 μm. As shown, many compounds of the conjugate binds with the captured target analyte. The conjugate is able to pass through the filter if unbound, but when bound to the target analyte, the conjugate remains captured to the filter membrane. With more bacterium, the concentration of labels is greater and thus produce an optically brighter target when excited. Finally, in FIG. 4C, the remaining labels 401 (i.e., the labels bound to target analyte 409) are excited by laser 411 and fluorescence from the labels 401 is captured by an optical detector 413. The optical detector 413 may then detect optical frequencies of the excited labels to determine concentration level of the target analyte directly from the membrane filter (i.e., direct membrane interrogation).

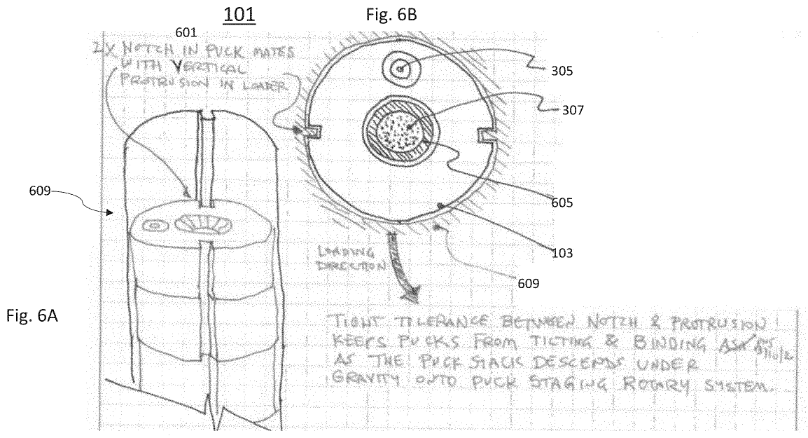

As shown in FIGS. 6A and 6B, testing cartridges 103, e.g., a puck containing a filter membrane, may include a notch 601 for storage, conjugate packet 305, membrane filter 307, and cone-shaped gasket 605 for mating with fluid paths 301. The notch 601 aids in preventing testing cartridges from tilting and binding to the side of the storage chute 609, i.e., puck loader. The conjugate packet 305, i.e., labelled antibodies, is positioned near a side wall to allow penetration by a piston from the side of the testing cartridge 103 for release of the conjugate into the membrane filter 307. The cone-shaped gasket 605 allows sealed capture of fluids from fluid paths 301.

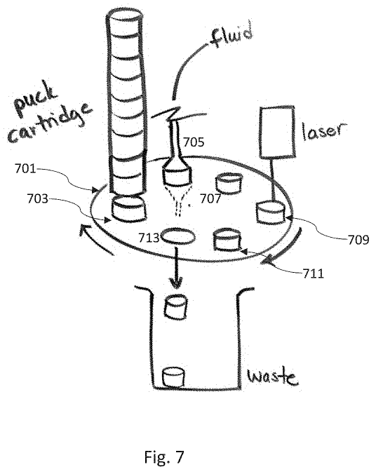

As shown in FIG. 7, in one embodiment of the invention, the testing cartridges 103 are moved from position to position by a rotating base 701. The rotating base 701 includes positions for testing cartridge storage drop position 703, fluid path position 705, optional drying position 707, excitation position 709, direct membrane interrogation position 711, and testing cartridge disposal position 713. At each position, the testing device 101 provides various actions which use the testing cartridges 103 to determine analyte levels in the water. A processor of the testing device 101 rotates base 701 to move to the next position in the rotation.

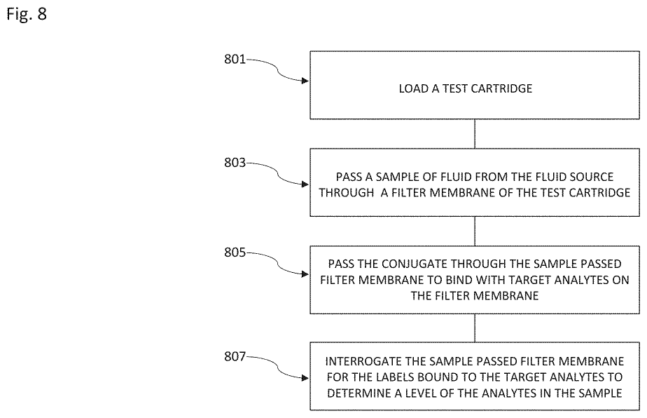

A method of using the testing device 101 with a testing cartridge 103 is shown in blocks 801-807 of FIG. 8. As described in block 801, the testing device 101, loads a test cartridge into a rotating base for positioning to pass fluids through the testing cartridge. The test cartridge may be dropped from a storage chute into a loaded position on the base. In block 803, the testing device 101 passes a sample of fluid from the fluid source through the filter membrane of the test cartridge. The cartridge may include a filter membrane that is configured to capture the targeted analyte, but allow passage of the conjugate. In block 805, the testing device 101 passes the conjugate of labels 401 and analyte-specific binding reagents 403 through the sample-passed filter membrane to bind with target analyte remaining on the filter membrane. In other words, in one embodiment, the labelled antibodies (i.e., conjugate), binds specifically with the target bacteria remaining on the filter membrane for later interrogation. In block 807, the testing device 101 directly interrogates the filter membrane for the remaining labels to determine a level of the target analyte in the sample. The testing device 101 provides the labels bound to the target analyte on the filter membrane with energy. By exciting the labels using a laser, based on the optical frequency of the excited labels, an optical detector of the testing device 101 may determine a level of target analyte in the sample directly from the membrane filter.

In one embodiment, the testing device 101 measures the level of Legionella pneumophila cells in tap water by using up-converting nanoparticles conjugated with anti-L. pneumophila antibodies. The water and conjugate are passed through a 25 mm thick PVDF filter membrane with 0.22 μm pores at 30 mL/min. Once the Legionella pneumophila and conjugate are captured on the filter membrane, the filter membrane is washed and may be dried prior to optical analysis.

The invention addresses design and ease of use difficulties of many previously available water testing systems. The invention provides an economical and easy to use platform when performing tests of water samples for analyte levels.

What is claimed is:

1. A method for testing a fluid source for a target analyte using a fluid analyte level assay device, the method comprising:
passing a sample of fluid from the fluid source through a filter membrane for the testing;
passing a conjugate of labels and analyte-specific binding reagents through the sample-passed filter membrane to bind with a target analyte captured on the filter membrane; and
interrogating the sample-passed filter membrane for the labels bound to the target analyte to determine a level of the target analyte in the sample.

2. The method of claim 1, further comprising:
loading a test cartridge including the filter membrane.

3. The method of claim 1, further comprising:
preparing the sample-passed filter membrane by washing the sample-passed filter membrane with solution.

4. The method of claim 3, wherein preparing the sample-passed filter membrane further comprising:
drying of the washed sample-passed filter membrane prior to interrogation.

5. The method of claim 1, wherein the sample is collected from a cooling tower.

6. The method of claim 1, wherein the labels are up-converting nanoparticles.

7. The method of claim 1, wherein determining the level of the target analyte includes exciting the labels and optically detecting the excited labels to determine the level of the target analyte.

8. The method of claim 1, wherein the target analyte is a bacteria or virus.

9. The method of claim 1, wherein the interrogation includes:
exciting the labels bound to the target analyte with a laser; and
optically detecting a fluorescence of the excited labels.

10. The method of claim 9, further comprising:
determining a level of the target analyte based upon an intensity of the optically detected fluorescence of the excited labels.

11. A fluid assay testing device configured to test a sample of fluid from a fluid source for analyte levels, the device comprising:
a fluid flow path to provide the sample of fluid to a test cartridge containing a filter membrane, and to provide a conjugate of analyte-specific binding reagents with labels to the test cartridge, wherein the conjugate collects on a filter membrane by specifically binding to a target analyte captured on the filter membrane of the test cartridge,
wherein the fluid flow path includes a pump and a valve;
a translational base to position a test cartridge for analysis;
an excitation mechanism to excite the labels for optical analysis; and
an optical detector to detect optical frequencies of the excited labels to determine a target analyte level.

12. The device of claim 11, further comprising:
a drying mechanism to dry the sample-passed test cartridge.

13. A fluid analyte testing system configured to test fluids for target analyte levels, the system comprising:
a test cartridge, wherein the test cartridge includes a filter membrane to collect the target analyte and one or more labels; and
a fluid analyte assay device to filter a sample of fluid from a fluid source through the test cartridge and directly detect the target analyte level based on the labels remaining on a filter membrane of the test cartridge.

14. The system of claim 13, wherein the test cartridge includes a plurality of testing sites to allow a plurality of tests of the fluid source from the same test cartridge.

15. The system of claim 13, wherein the labels are conjugated with analyte-specific binding reagents for reacting to the target analyte, wherein the labels absorb energy to emit light.

16. The system of claim 15, wherein the labels are selected from the group consisting of colorimetric elements, phosphor molecules, and up-converting nanoparticles.

17. The system of claim 13, wherein the assay device includes gated fluidic paths for water, conjugates, or reagents to be filtered through the test cartridge.

18. The system of claim 13, wherein the assay device directly detects the target analyte captured on the filter membrane using an optical reader.

19. The system of claim 13, wherein the fluid analyte testing system is a water testing system.
